(19) United States
(12) Patent Application Publication
(10) Pub. No.: US 2010/0325073 A1
(43) Pub. Date: Dec. 23, 2010

(54) NITROGEN OXIDE SENSITIVE FIELD EFFECT TRANSISTORS FOR EXPLOSIVE DETECTION COMPRISING FUNCTIONALIZED NON-OXIDIZED SILICON NANOWIRES

(75) Inventor: Hossam Haick, Haifa (IL)

Correspondence Address: KEVIN D. MCCARTHY;ROACH BROWN MCCARTHY & GRUBER, P.C., 424 MAIN STREET, 1920 LIBERTY BUILDING, BUFFALO, NY, 14202, US

(73) Assignee: TECHNION RESEARCH AND DEVELOPMENT FOUNDATION LTD., Haifa (IL)

(21) Appl. No.: 12/867,258

(22) PCT Filed: Feb. 18, 2009
(86) PCT No.: PCT/IL09/00185

§ 371 Date: Aug. 12, 2010

(30) Foreign Application Priority Data
Feb. 18, 2008 (IL) 189576

(51) Int. Cl. G06F 15/18; H01L 29/772

(52) U.S. Cl. 706/12; 257/253; 257/E29.242; 706/18

(57) ABSTRACT

An apparatus for detecting volatile compounds derived from explosive materials with very high sensitivity. The apparatus is composed of field effect transistors of non-oxidized silicon nanowires modified with specific functional groups including, in particular, amine, imine and/or carboxyl moieties. Further a system is provided comprising the apparatus in conjunction with learning and pattern recognition algorithms and methods of use thereof for detecting and quantifying specific explosive compounds.

### FIELD OF THE INVENTION

The present invention relates to an electronic device comprising chemically sensitive field effect transistors of non-oxidized, functionalized silicon nanowires for detecting explosive materials.

### BACKGROUND OF THE INVENTION

The hitherto known methods for detecting explosive materials are mainly directed towards the detection of nitrogen containing compounds. These methods usually require concentrating vapors of explosive nitro-compounds followed by their decomposition to produce gases of nitric oxide (NO) and/or nitric dioxide (NO₂). These nitric based gases can subsequently be detected using a variety of techniques including gas, capillary electrophoresis and high performance liquid chromatography; mass spectrometry; and ion mobility analyzer. U.S. Pat. Nos. 5,092,218; 5,109,691; 6,571,649; and 6,840,120 disclose exemplary uses of said techniques for explosive detection.

Other commonly used techniques include x-ray scattering, neutron analysis, nuclear quadrupole resonance, FTIR and Raman spectrometry, and immunoassays (Wang, Analy. Chimi. Acta, 2004, 507: 3-10). U.S. Pat. No. 5,801,297 discloses methods and devices for the detection of odorous substances including explosives comprising a plurality of gas sensors selected from semiconductor gas sensors, conductive polymer gas sensors, and acoustic surface wave gas sensors. U.S. Pat. No. 6,872,786 discloses a molecularly imprinted polymeric explosives sensor, which possesses selective binding affinity for explosives. U.S. Pat. No. 5,585,575 discloses an explosive detection screening system which comprises a concentration and analyzing system for the purification of the collected vapor and/or particulate emissions and their subsequent detailed chemical analysis. U.S. Pat. No. 7,224,345 discloses a system for electrochemical detection based on carbon or carbon/gold working electrode having a modified surface to detect trace amounts of nitro-aromatic compounds.

The most frequently used sensing devices for detecting explosive materials are based on the lock-and-key approach, wherein each sensor detects one explosive material. In this manner, the sensors are designed to detect very specific target molecules. Hence, the applicability of these sensors is limited.

Electronic nose devices perform odor detection through the use of an array of cross-reactive sensors in conjunction with pattern recognition algorithms. In contrast to the “lock-and-key” model, each sensor in the electronic nose device is widely responsive to a variety of odorants. In this architecture, each analyte produces a distinct signature from the array of broadly cross-reactive sensors. This configuration allows to considerably widen the variety of compounds to which a given matrix is sensitive, to increase the degree of component identification and, in specific cases, to perform an analysis of individual components in complex multi-component mixtures. Pattern recognition algorithms can then be applied to the entire set of signals, obtained simultaneously from all the sensors in the array, in order to glean information on the identity, properties and concentration of the vapors exposed to the sensor array.

Sensor devices for detecting and analyzing volatile compounds including volatile compounds derived from vapors of explosives are disclosed in e.g. U.S. Pat. Nos. 7,469,076, 6,841,391, 6,839,636, 6,820,012, 6,767,732, 6,703,241, 6,620,109, 6,609,068, 6,606,566, 6,467,333, 6,411,905, 6,319,724, in U.S. Patent Application Nos. 2006/0231420, and 2001/0041366 and in Toal and Trogler (J. Mater. Chem., 2006, 16: 2871-2883). A transition metal oxide gas sensor is described in U.S. Pat. No. 6,173,602. A sensor of complementary metal oxide semiconductor field effect transistor is described in Stern et al. (Nature, 2007, 445: 519-522).

The use of silicon nanowire field effect transistors (Si NW FETs) for detecting volatile compounds has been employed. Oxide-coated Si NW FETs were modified with amino siloxane functional groups to impart high sensitivity towards pH (Patolsky and Lieber, Mater. Today, 2005, 8: 20-28). The Si NW field effect transistors were further modified with a variety of biological receptors to selectively detect biological species in solution. International patent application WO 2008/030395 discloses a nanoelectronic device for detecting target molecules, comprising: an array of nanowires serving as sensors of target molecules, the nanowires comprising (i) electrically contacted regions at their ends, the electrically contacted regions being covered with an insulating material and (ii) a central window region coated with a probe molecule; and a microfluidics channel placed across the array of silicon nanowires, the microfluidics channel adapted to direct a flow of solution containing the target molecules.

International patent application WO 2005/004204 discloses a method by which silicon nanostructures may be selectively coated with molecules or biomolecules using an electrochemical process. Further disclosed are applications toward the fabrication of molecular electronic circuitry and nanoelectronic molecular sensor arrays. In particular, WO 2005/004204 teaches that in order to utilize the silicon nanowires as electrochemical electrodes, and to maximize the sensitivity of a silicon nanowire molecular electronic sensor device, it is desirable to remove the silicon oxide (SiO₂) insulating layer, thus enabling the binding of the precursor molecule directly on the Si nanowire conductor.

Oxide-coating of a Si NW is believed to induce trap states at the Si/Si-oxide interface thus acting as a dielectric layer. This in turn lowers and consequently limits the effect of gate voltage on the transconductance of Si NW field effect transistors. This limitation affects the response of sensors based on oxide-coated Si NW field effect transistors to their environment. In a typical SiO₂-coated Si NW field effect transistor, the transconductance responds weakly to the applied gate voltage, V_{g}, where conductivity changes by two orders of magnitude between V_{g}=−5V and V_{g}=+5 V, with no significant on/off state transition within this gate-bias region. This behavior is compatible with the characteristics of oxidized Si wherein both the Si/SiO₂ interface and the SiO₂ surface defects trap and scatter carriers, and as a result, decrease the effect of V_{g} (Lupke, Surf Sci. Rep., 1999, 35:75-161). On the contrary, devices that are based on non-oxidized Si NWs as well as those based on macroscopic planar Si (111) surfaces, exhibit low interface state density. Yet, non-oxidized Si NWs as well as Si surfaces that are terminated with hydrogen tend to undergo oxidation upon exposure to ambient conditions, resulting in the formation of defects in the sensors.

It has been reported by the inventor of the present invention, that Si NW modified by covalent Si—CH₃ functionality, show atmospheric stability, high conductance values, and low surface defect levels. These methyl functionalized Si NWs were shown to form air-stable Si NW FETs having on-off ratios in excess of 10⁵ over a relatively small gate voltage swing (±2 V) (Haick et al., J. Am. Chem. Soc., 2006, 128: 8990-8991). However, exposure of these methyl-functionalized devices to analytes barely provides sensing responses, most probably due to the low ability of the methyl groups to adsorb vapor/liquid analytes. Other modifications of Si NW surfaces are described in Puniredd et al. (J. Am. Chem. Soc., 2008, 130: 13727-13734) and Assad et al. (J. Am. Chem. Soc., 2008, 130: 17670-17671).

International patent application WO 2009/013754 to the inventor of the present invention discloses an electronic device comprising chemically sensitive field effect transistors of non-oxidized, functionalized silicon nanowires for detecting volatile organic compounds and methods of use thereof in diagnosing diseases including various types of cancer.

Bumenovich et al. (J. Am. Chem. Soc., 2006, 128: 16323-16331) reported that Si NWs without the native oxide exhibit improved solution-gated filed-effect transistor characteristics and a significantly enhanced sensitivity to single stranded DNA detection, with an accompanying two orders of magnitude improvement in the dynamic range of sensing.

Mcalpine et al. (Nature Mater., 2007, 6(5): 379-384) discloses the use of Si NW FETs as sensors which exhibits parts-per-billion sensitivity to NO₂. Notwithstanding these recent successes, the detection of explosives through air requires a significantly higher sensitivity which is often met by pre-concentrating the explosive vapors prior to measurement thus leading to lengthier measurements. Real-time measurement of minute quantities of explosive vapors remains a challenge.

Thus, there is an unmet need for a highly sensitive reliable device to detect minute concentrations of explosives through air.

### SUMMARY OF THE INVENTION

The present invention provides an apparatus for detecting volatile compounds released from explosive materials with very high sensitivity. The apparatus disclosed herein comprises field effect transistors of non-oxidized functionalized silicon nanowires (Si NW FETs) wherein the nanowires are modified with unique compositions of functional groups comprising amine, imine, amide, ammonium, keto, alcohol, phosphate, thiol, sulfonate, sulfonyl and/or carboxyl derivatives. Within the scope of the present invention is a system comprising the apparatus in conjunction with learning and pattern recognition algorithms which receive sensor output signals and compare them to stored data. Methods of preparing the apparatus and methods of use thereof for detecting and quantifying specific explosive compounds are disclosed.

The invention is based in part on the unexpected finding that sensors of non-oxidized silicon nanowires modified with unique compositions of amine, imine, amide, ammonium, keto, alcohol, phosphate, thiol, sulfonate, sulfonyl and/or carboxyl functional groups provide improved sensing of explosive materials. The lack of oxide layer on the surface of the nanowires as well as the modifying functional groups, provide enhanced selectivity towards volatile explosives. Improved sensitivity and selectivity thus enable the detection of minute quantities of volatile explosive compounds preferably without pre-concentrating the explosive vapors prior to measurement.

According to a first aspect the present invention provides an apparatus for detecting volatile compounds derived from explosive materials, comprising at least one chemically sensitive sensor comprising field effect transistors (FETs) of non-oxidized, silicon nanowires (Si NW) functionalized with at least one of an amine, an imine, an amide, an ammonium, a keto, an alcohol, a phosphate, a thiol, a sulfonate, a sulfonyl or a carboxyl moiety.

According to another aspect, the present invention provides a system comprising i) an apparatus for detecting volatile compounds derived from explosive materials, wherein the apparatus comprises an array of chemically sensitive sensors comprising field effect transistors (FETs) of non-oxidized, silicon nanowires (Si NW) functionalized with at least one of an amine, an imine, an amide, an ammonium, a keto, an alcohol, a phosphate, a thiol, a sulfonate, a sulfonyl or a carboxyl moiety; and ii) learning and pattern recognition analyzer wherein the learning and pattern recognition analyzer receives sensor signal outputs and compares them to stored data.

In one embodiment, the apparatus and system of the present invention detect volatile compounds derived from explosive materials with sensitivity below one part per million (ppm). In another embodiment, the apparatus and system of the present invention detect volatile compounds derived from explosive materials with sensitivity of less than 100 parts per billion (ppb). In yet another embodiment, the apparatus and system disclosed herein detect volatile compounds derived from explosive materials with sensitivity of one part per billion (ppb), or less.

In some embodiments, the Si NW FETs are manufactured in a top-down approach. In alternative embodiments, the Si NW FETs are manufactured in a bottom-up approach.

In particular embodiments, the functional groups which are used to modify the surface of the nanowires include, but are not limited to: carboxyalkyl, carboxycycloalkyl, carboxyalkenyl, carboxyalkynyl, carboxyaryl, carboxyheterocyclyl, carboxyheteroaryl, carboxyalkylaryl, carboxyalkylalkenyl, carboxyalkylalkynyl, carboxyalkylcycloalkyl, carboxyalkylheterocyclyl carboxyalkylheteroaryl, alkylamine, cycloalkylamine, alkenylamine, alkynylamine, arylamine, heterocyclylamine, heteroarylamine, alkylarylamine, alkylalkenylamine, alkylalkynylamine, alkylcycloalkylamine, alkylheterocyclylamine alkylheteroarylamine, alkylimine, cycloalkylimine, alkenylimine, alkynylimine, arylimine, heterocyclylimine, heteroarylimine, alkylarylimine, alkylalkenylimine, alkylalkynylimine, alkylcycloalkylimine, alkylheterocyclylimine, alkylheteroarylimine and combinations and derivatives thereof.

In currently preferred embodiments, the functional groups which are used to modify the surface of the nanowires include, but are not limited to, ethyleneimine, aniline-boronic acid, diethyl ester, 2,5-dimercaptoterephthalic acid, n-(3-trifluoroethanesulfonyloxypropyl)-anthraquinone-2-carboxamide, thiophene, 1-[4-(4-dimethylamino-phenylazo)-3-[3,5-bis[3,5-bis [3,5-bis(3-butene-1-oxy)benzyloxy]benzyloxy]benzyloxy]phenyl]-2,2,2 trifluoroethanone, permethylated α-cyclodextrin-6^{A}-monoalcohol nitrate, dinitrophenyl substituted β-cyclodextrin, β- and γ-CD bearing a 4-amino-7-nitrobenz-2-oxa-1,3-diazole, sulfated and carboxymethylated β-cyclodextrins, mono(6-cyclohexylamino-6-deoxy)-β-cyclodextrin, mono(6-benzyl-imino-6-deoxy)-β-cyclodextrin, mono[6-(o-aminophenyl)imino-6-deoxy]-β-cyclodextrin, mono[6-(p-aminophenyl)imino-6-deoxy]-β-cyclodextrin, mono [6-(α-naphthyl)imino-6-deoxy]-β-cyclodextrin, hexakis(6-O-benzoyl)-α-cyclodextrin, heptakis(2,3,6-tri-O-benzoyl)-β-cyclodextrin, hexakis(2,3-di-O-benzyl)-α-cyclodextrin, hexakis(6-O-benzoyl-2,3-di-O-benzyl)-α-cyclodextrin, 2- and 6-amino-β-cyclodextrin, and 2A,3A-alloepithio-2A,3A-dideoxy-β-cyclodextrin; and combinations thereof. In currently preferred embodiments, the functional groups which are used to modify the surface of the nanowires are selected from the group consisting of 4-(3-trifluoromethylazirino)benzoyl-N-succinimide (TDBA-OSu), para-phenylenediamine (PPD) and a combination thereof.

In certain embodiments, the surface of the nanowires is modified with a thin polymer film selected from poly(3,4-ethylenedioxy)-thiophene-poly(styrene sulfonate) (PEDOT-PSS), poly(sulfone), poly(ethylene-co-vinyl acetate), poly(methyl methacrylate), tributyl phosphate (TBP), tricresyl phosphate, polyaniline, poly(vinylpyrrolidone), polycaprolactone, hydroxypropylcellulose, poly(ethyleneimine), tetracosanoic acid, tetraoctylammonium bromide, lauric acid, propyl gallate, quinacrine dihydrochloride dehydrate, and quinacrine dihydrochloride. According to currently preferred embodiments, the polymer films have thicknesses ranging from about 1 nm to about 500 nm.

In some embodiments, the apparatus and system of the present invention detect minute concentration of explosive materials selected from the group consisting of pentaerythitol tetranitrate (PETN), tetranitro-tetrazacylooctane (HMX), nitroglycerin (NG), ethylene glycol dinitrate (EGDN), NH₄NO₃, dinitrotoluene (DNT), trinitrotoluene (TNT), tetryl, picric acid, and cyclotrimethylenetrinitramine (RDX). In particular embodiments, the volatile compounds derived from explosive materials are selected from NO and NO₂ gases.

According various embodiments, the system of the present invention comprises a learning and pattern recognition analyzer. The learning and pattern recognition analyzer may utilize various algorithms including, but not limited to, algorithms based on artificial neural networks, multi-layer perception (MLP), generalized regression neural network (GRNN), fuzzy inference systems (FIS), self-organizing map (SOM), radial bias function (RBF), genetic algorithms (GAS), neuro-fuzzy systems (NFS), adaptive resonance theory (ART) and statistical methods such as principal component analysis (PCA), partial least squares (PLS), multiple linear regression (MLR), principal component regression (PCR), discriminant function analysis (DFA) including linear discriminant analysis (LDA), cluster analysis including nearest neighbor, and the like.

According to another aspect, the present invention provides a method of determining at least one of the composition and concentration of volatile compounds derived from explosive materials in a sample, comprising the steps of: (a) providing a system comprising an apparatus comprising an array of chemically sensitive sensors comprising field effect transistors (FETs) of non-oxidized silicon nanowires (Si NWs) functionalized with at least one of an amine, an imine, an amide, an ammonium, a keto, an alcohol, a phosphate, a thiol, a sulfonate, a sulfonyl or a carboxyl moiety, and a learning and pattern recognition analyzer, wherein the learning and pattern recognition analyzer receives sensor output signals from the apparatus and compares them to stored data, (b) exposing the sensor array of the apparatus to the sample, and (c) using pattern recognition algorithms to detect the presence of volatile compounds derived from explosive materials in the sample.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIG. 1 is a schematic representation of a Si NW field effect transistor arrangement used for chemical sensing without a reference electrode. The molecular layer is directly bonded to the semiconductor and the gating is done from the back. ‘V’ represents volatile compounds derived from explosive materials, and ‘S’ represents sensing molecules.

FIG. 2 is a schematic diagram illustrating the differentiation between odorants using an array of broadly-cross reactive sensors, in which each individual sensor responds to a variety of odorants, in conjugation with pattern recognition algorithms to allow classification. ‘A’—raw measurements, ‘B’—normalized measurements, ‘C’—feature vector, ‘D’—odor class (confidence level), ‘E’—post processed odor class, ‘F’—decision making, ‘G’—classification, ‘H’—dimensionality reduction, and ‘I’—signal preprocessing.

FIG. 3 is a schematic representation of the subsequent functionalization of the CH₃—CH═CH—Si NW with photoactive aryldiazirine crosslinker.

FIGS. 4A-4B are X-ray Photoelectron Spectroscopy (XPS) of a propenyl-terminated Si NWs before (diamonds) and after functionalization with 4-(3-trifluoromethylazirino)benzoyl-N-succinimide (TDBA-OSu; squares) and further functionalization with para-phenylenediamine (PPD; triangles) at 350-800 eV (4A) and 390-415 eV (4B).

FIG. 5 is the absolute response of TDBA-OSu-CH₂—CH═CH—Si NW FETs (denoted S1) and PPD-TDBA-OSu-CH₂—CH═CH—Si NW FETs (denoted S2) to 10 ppb of NO and NO₂.

### DETAILED DESCRIPTION OF THE INVENTION

The present invention provides an apparatus for detecting volatile compounds released by explosive materials, with very high sensitivity. The invention further provides a system comprising an array of sensors and pattern recognition algorithms, including principal component analysis and neural networks, to detect and classify a wide variety of explosive vapors. According to the principles of the present invention, the apparatus comprises chemically sensitive field effect transistors (FETs) of non-oxidized, functionalized silicon nanowires wherein the nanowires are modified with unique compositions of functional groups comprising amine, imine, amide, ammonium, keto, alcohol, phosphate, thiol, sulfonate, sulfonyl and/or carboxyl derivatives. Further provided are methods of use thereof in detecting explosives.

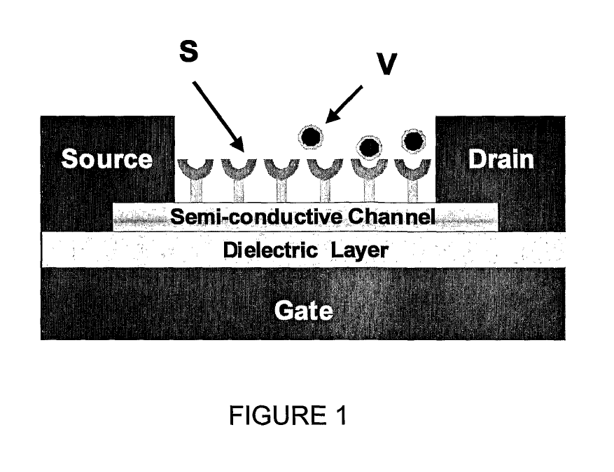

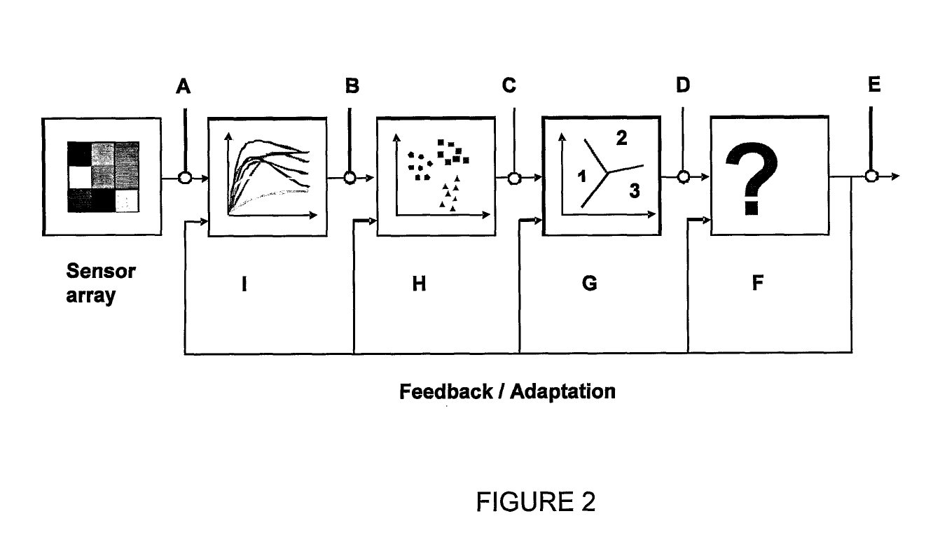

The apparatus and system disclosed herein comprise chemically sensitive field effect transistors (FETs) of non-oxidized silicon nanowires functionalized with moieties selected from amine, imine, amide, ammonium, keto, alcohol, phosphate, thiol, sulfonate, sulfonyl and carboxyl, and combinations thereof (FIG. 1). Sensing is obtained through adsorption of vapors to provide changes in electrical resistance. The electrical signals are then conveyed to a pattern recognition analyzer to generate qualitative identification and preferably quantitative analysis of desired volatile compounds. A schematic diagram of the differentiation between odorants using the electronic nose devices is illustrated in FIG. 2. The array of sensors is exposed to a variety of volatiles to provide an electronic response vs. time (2^{nd} box on the left). The dimensionality is then reduced wherein the data is represented by a new basis set (f₂ vs. f₁; 3^{rd} box on the left). This representation allows to classify the different odors (1, 2 & 3; 4^{th} box on the left). The procedure can be iteratively performed until satisfactory odor classification is achieved.

Similar to olfactory receptors, increased sensitivity as well as on/off rates of chemical sensors is typically achieved by reducing the dimensions of the sensing material. Chemical sensors based on nanomaterials are thus more sensitive, more controlled, and more suitable to differentiate between subtle differences in mixtures of volatile compounds. This feature is significantly important in instances wherein identification of a specific explosive is required. Silicon nanowires (Si NW) devoid of the native oxide layer and further modified with particular amine, imine, amide, ammonium, keto, alcohol, phosphate, thiol, sulfonate, sulfonyl and/or carboxyl moieties offer unique opportunities for signal transduction associated with selective recognition of explosive compounds of interest.

In order to detect explosives, the Si NW FETs sensors of the present invention are designed to adsorb compounds which are mostly polar in nature. The sensors are therefore functionalized with either one of an amine, an imine, an amide, an ammonium, a keto, an alcohol, a phosphate, a thiol, a sulfonate, a sulfonyl or a carboxyl moiety in order to possess high affinity towards explosive compounds and vapors derived from explosive materials. Within the scope of the present invention is the detection of explosives including, but are not limited to, pentaerythitol tetranitrate (PETN), tetranitro-tetrazacylooctane (HMX), nitroglycerin (NG), ethylene glycol dinitrate (EGDN), NH₄NO₃, o-nitrotoluene (2NT), m-nitrotoluene (3NT), p-nitrotoluene (4NT), dinitrotoluene (DNT), amino-dinitrotoluene (Am-DNT), trinitrotoluene (TNT), trinitrobenzene (TNB), dinitrobenzene (DNB), nitrobenzene (NB), methyl-2,4,6-trinitrophenylnitramine (Tetryl), picric acid, cyclotrimethylenetrinitramine (RDX), combinations and mixtures thereof. Examples of explosive mixtures are listed in Table 1.

TABLE 1
Typical mixtures of common explosive materials

| Explosive Mixture | Main composition |
|---|---|
| C-2 | RDX + TNT + DNT + NG |
| C-3 | RDX + TNT + DNT + Tetryl + NG |
| Cyclotol | RDX + TNT |
| Pentolite | PETN + TNT |
| PTX-1 | RDX + TNT + Tetryl |
| PTX-2 | RDX + TNT + PETN |
| Tetryol | TNT + Tetryl |
| Semtex-H | PETN + RDX |

### Device

The apparatus according to the principles of the present invention uses at least one sensor of surface-modified, non-oxidized Si NW FET. In another embodiment the apparatus uses finely-tuned arrays of surface-modified, non-oxidized Si NW FET sensors. The array of sensors comprises a plurality of sensors between 2 to 1000 sensors, more preferably between 2 to 500 sensors, even more preferably between 2 to 250 sensors, and most preferably between 2 to 125 sensors in an array.

As used herein, the term nanowire refers to any elongated conductive or semiconductive material that includes at least one cross sectional dimension that is less than 500 nm, and has an aspect ratio (length:width) of greater than 10, preferably, greater than 50, and more preferably, greater than 100. In specific embodiments each nanowire has diameter of 2-120 nm, wherein the nanowires have a cylinder-like shape with a circle-like cross section, or equivalent dimensions wherein the nanowires have other cross sectional shapes including, but not limited to, trapezoidal, triangular, square, or rectangular. Si NWs having diameters (or equivalent dimensions for shapes other than cylinder) larger than 120 nm possess electrical/physical properties similar to planar Si. Si NWs with diameters (or equivalent dimensions for shapes other than cylinder) less than 2 nm consist mostly of SiO₂, with very low percentage of Si core. Thus the Si NWs whose diameter exceeds the 2-120 nm range, are less suitable for sensing applications in accordance with the present invention. Without being bound by theory or mechanism of action, elimination of the intervening oxide layer from the Si NW FETs provides increased sensitivity. The chemical modification of the surface to incorporate amine, imine, amide, ammonium, keto, alcohol, phosphate, thiol, sulfonate, sulfonyl and/or carboxyl moieties provides stable Si NW surfaces even upon exposure to air and/or humidity, and further endows the Si NWs with chemical inertness and good electronic properties due to the passivation of Si NW surface states. Using the two step chlorination/alkylation process (Webb et al., J. Phys. Chem. B, 2003, 107(23): 5404-5412), 50-100% coverage of the Si NW surface sites is obtained. This coverage provides high density functionalities which allow better signal/noise ratios. The modifications of the Si NW surfaces can be tailor-made to control the electrical properties of the Si NWs (by, for example, utilizing adsorptive molecular dipoles on the Si NW surface, applying back gate voltage, and/or use of four-probe configuration), the contact resistance between the Si NWs and further allows the elimination of the electrodes, thus achieving the required sensitivity for detecting explosive vapors.

Formation of the Si NW FETs. The non-oxidized Si NW FET-based sensors of the present invention can be manufactured in two different manners: a bottom-up approach or a top-down approach.

In one embodiment of the invention Si NW FETs sensors are manufactured through a bottom-up approach. Si NWs that are grown by, for example, vapor-liquid-solids, chemical vapor deposition (CVD), or oxide-assisted growth are dispersed from organic solvent (e.g., isopropanol or ethanol) onto a doped Si substrate containing a thin film of dielectric layer (e.g., SiO₂, ZrO₂, etc.). The deposited Si NWs can be “bare” or “as-synthesized” ones, namely with oxide layer and/or without modifying monolayer of organic molecules. Alternatively, the deposited Si NW can be non-oxidized possessing a variety of functional groups. The source/drain contacts to the Si NWs are defined by electron beam lithography followed by evaporation of a metal to form an ohmic contact. The latter can also be performed through focused ion beam (FIB), or using contact printing. The devices are then annealed to improve the quality of the contacts. The term “functionalized Si NW” as used herein refers to a continuous or discontinuous monolayer (or multilayers) of molecules that coat the surface of Si NW. The term non-oxidized as used herein refers to the removal of the native oxide layer by methods well known to a skilled artisan. According to the principles of the present invention, the functional groups are attached to the Si atop sites with a direct covalent bond.

In another embodiment, the sensors are manufactured through a top-down approach. The fabrication process starts from a SOI-STMOX wafer, with thin top silicon layer, isolated from the silicon substrate by a buried silicon dioxide layer. Mask definition is performed by means of high resolution e-beam lithography. A bilayer of polymethylmethacrylate (PMMA) composed of two polymers with different lithography characteristics is used. The bottom layer is characterized by a copolymer which has both minor molecular weight and higher susceptibility than the upper PMMA layer. The exposure is performed using e-beam lithography with an acceleration voltage of 30 kV. The PMMA resistance is then developed in a solution of MiBK and isopropyl alcohol (IPA) in a ratio of 1:3 respectively. The pattern is transferred from the PMMA to the top of the SiO₂ layer by BHF etching. The central region, where the silicon is defined, is linked through small connections to the device leads. A 35 wt % KOH solution, saturated with IPA, is used. Following this process, a nanowire is formed in the central region.

Surface modification of the Si NW FETs. The addition of chemical functionalities to the nanowires, whether before or after integration in the FET device, is performed through the use of reagents having different backbones and functional groups. Desired reagents are synthesized and attached to the Si NW surfaces. The functional groups used comprise at least one of an amine, an imine, an amide, an ammonium, a keto, an alcohol, a phosphate, a thiol, a sulfonate, a sulfonyl or a carboxyl moiety including, but are not limited to, carboxyalkyl, carboxycycloalkyl, carboxyalkenyl, carboxyalkynyl, carboxyaryl, carboxyheterocyclyl, carboxyheteroaryl, carboxyalkylaryl, carboxyalkylalkenyl, carboxyalkylalkynyl, carboxyalkylcycloalkyl, carboxyalkylheterocyclyl carboxyalkylheteroaryl, alkylamine, cycloalkylamine, alkenylamine, alkynylamine, arylamine, heterocyclylamine, heteroarylamine, alkylarylamine, alkylalkenylamine, alkylalkynylamine, alkylcycloalkylamine, alkylheterocyclylamine alkylheteroarylamine, alkylimine, cycloalkylimine, alkenylimine, alkynylimine, arylimine, heterocyclylimine, heteroarylimine, alkylarylimine, alkylalkenylimine, alkylalkynylimine, alkylcycloalkylimine, alkylheterocyclylimine, alkylheteroarylimine and combinations and derivatives thereof.

An “alkyl” group refers to a saturated aliphatic hydrocarbon, including straight-chain, branched-chain and cyclic alkyl groups. In one embodiment, the alkyl group has 1-12 carbons designated here as C₁-C₁₂-alkyl. In another embodiment, the alkyl group has 1-6 carbons designated here as C₁-C₆-alkyl. In another embodiment, the alkyl group has 1-4 carbons designated here as C₁-C₄-alkyl. The alkyl group may be unsubstituted or substituted by one or more groups selected from halogen, haloalkyl, acyl, amido, ester, cyano, nitro, and azido.

A “cycloalkyl” group refers to a non-aromatic mono- or multicyclic ring system. In one embodiment, the cyclo-alkyl group has 3-10 carbon atoms. In another embodiment, the cyclo-alkyl group has 5-10 carbon atoms. Exemplary monocyclic cycloalkyl groups include cyclopentyl, cyclohexyl, cycloheptyl and the like. An alkylcycloalkyl is an alkyl group as defined herein bonded to a cycloalkyl group as defined herein. The cycloalkyl group can be unsubstituted or substituted with any one or more of the substituents defined above for alkyl.

An “alkenyl” group refers to an aliphatic hydrocarbon group containing a carbon-carbon double bond including straight-chain, branched-chain and cyclic alkenyl groups. In one embodiment, the alkenyl group has 2-8 carbon atoms. In another embodiment, the alkenyl group has 2-4 carbon atoms in the chain. Exemplary alkenyl groups include ethenyl, propenyl, n-butenyl, i-butenyl, 3-methylbut-2-enyl, n-pentenyl, heptenyl, octenyl, cyclohexyl-butenyl and decenyl. An alkylalkenyl is an alkyl group as defined herein bonded to an alkenyl group as defined herein. The alkenyl group can be unsubstituted or substituted through available carbon atoms with one or more groups defined hereinabove for alkyl.

An “alkynyl” group refers to an aliphatic hydrocarbon group containing a carbon-carbon triple bond including straight-chain and branched-chain. In one embodiment, the alkynyl group has 2-8 carbon atoms in the chain. In another embodiment, the alkynyl group has 2-4 carbon atoms in the chain. Exemplary alkynyl groups include ethynyl, propynyl, n-butynyl, 2-butynyl, 3-methylbutynyl, n-pentynyl, heptynyl, octynyl and decynyl. An alkylalkynyl is an alkyl group as defined herein bonded to an alkynyl group as defined herein. The alkynyl group can be unsubstituted or substituted through available carbon atoms with one or more groups defined hereinabove for alkyl.

An “aryl” group refers to an aromatic monocyclic or multicyclic ring system. In one embodiment, the aryl group has 6-10 carbon atoms. The aryl is optionally substituted at least one “ring system substituents” and combinations thereof, and are as defined herein. Exemplary aryl groups include phenyl or naphthyl. An alkylaryl is an alkyl group as defined herein bonded to an aryl group as defined herein. The aryl group can be unsubstituted or substituted through available carbon atoms with one or more groups defined hereinabove for alkyl.

A “heteroaryl” group refers to a heteroaromatic system containing at least one heteroatom ring wherein the atom is selected from nitrogen, sulfur and oxygen. The heteroaryl contains 5 or more ring atoms. The heteroaryl group can be monocyclic, bicyclic, tricyclic and the like. Also included in this definition are the benzoheterocyclic rings. Non-limiting examples of heteroaryls include thienyl, benzothienyl, 1-naphthothienyl, thianthrenyl, furyl, benzofuryl, pyrrolyl, imidazolyl, pyrazolyl, pyridyl, pyrazinyl, pyrimidinyl, pyridazinyl, indolyl, isoindolyl, indazolyl, purinyl, isoquinolyl, quinolyl, naphthyridinyl, quinoxalinyl, quinazolinyl, cinnolinyl, pteridinyl, carbolinyl, thiazolyl, oxazolyl, isothiazolyl, isoxazolyl and the like. The heteroaryl group can be unsubstituted or substituted through available atoms with one or more groups defined hereinabove for alkyl.

A “heterocyclic ring” or “heterocyclyl” group refers to a five-membered to eight-membered rings that have 1 to 4 heteroatoms, such as oxygen, sulfur and/or in particular nitrogen. These five-membered to eight-membered rings can be saturated, fully unsaturated or partially unsaturated, with fully saturated rings being preferred. Preferred heterocyclic rings include piperidinyl, pyrrolidinyl pyrrolinyl, pyrazolinyl, pyrazolidinyl, morpholinyl, thiomorpholinyl, pyranyl, thiopyranyl, piperazinyl, indolinyl, dihydrofuranyl, tetrahydrofuranyl, dihydrothiophenyl, tetrahydrothiophenyl, dihydropyranyl, tetrahydropyranyl, and the like. An alkylheterocyclyl is an alkyl group as defined herein bonded to a heterocyclyl group as defined herein. The heterocyclyl group can be unsubstituted or substituted through available atoms with one or more groups defined hereinabove for alkyl.

“Ring system substituents” refer to substituents attached to aromatic or non-aromatic ring systems including, but not limited to, H, halo, haloalkyl, (C₁-C₈)alkyl, (C₂-C₈)alkenyl, (C₂-C₈)alkynyl, (C₆-C₁₀)aryl, acyl, amido, ester, cyano, nitro, azido, and the like.

A “halogen” or “halo” group refers to chlorine, bromine, fluorine, and iodine. The term “haloalkyl” refers to an alkyl group having some or all of the hydrogens independently replaced by a halogen group including, but not limited to, trichloromethyl, tribromomethyl, tifluoromethyl, triiodomethyl, difluoromethyl, chlorodifluoromethyl, pentafluoroethyl, 1,1-difluoroethyl bromomethyl, chloromethyl, fluoromethyl, iodomethyl, and the like.

An “amine” moiety refers to an —NRR′ group, wherein R and R′ are independently selected from hydrogen, alkyl and aryl. A currently preferred amine group is —NH₂. An “alkylamine” group is an alkyl group as defined herein bonded to an amine group as defined herein.

An “imine” moiety refers to an —NRR′ group containing a carbon-nitrogen double bond wherein R and R′ are independently selected from hydrogen, alkyl and aryl. An “alkylimine” group is an alkyl group as defined herein bonded to an imine group as defined herein.

An “amide” moiety refers to a —C(O)NRR′ group wherein R and R′ are independently selected from hydrogen, alkyl and aryl. An “alkylamide” group is an alkyl group as defined herein bonded to an amide group as defined herein.

An “ammonium” moiety refers to —NH₄⁺ group.

An “acyl” moiety encompasses groups such as, but not limited to, formyl, acetyl, propionyl, butyryl, pentanoyl, pivaloyl, hexanoyl, heptanoyl, octanoyl, nonanoyl, decanoyl, undecanoyl, dodecanoyl, benzoyl and the like. Currently preferred acyl groups are acetyl and benzoyl.

A “thio” or “thiol” moiety refers to —SH group or, if between two other groups, —S—. A “thioalkyl” group is an alkyl group as defined herein bonded to a thiol group as defined herein.

A “sulfonyl” or “sulfone” moiety refers to —S(O)₂— group. An “alkylsulfone” group is an alkyl group as defined herein bonded to a sulfonyl group as defined herein.

A “sulfonate” moiety refers to a —S(O)₂O— group.

A “carboxy” or “carboxyl” moiety refers carboxylic acid and derivatives thereof including in particular, ester derivatives and amide derivatives. A “carboxyalkyl” group is an alkyl group as defined herein bonded to a carboxy group as defined herein.

A “keto” moiety refers to a —C(O)— group.

An “alcohol” moiety refers to an —OH group including in particular sugar alcohols (cyclodextrin) and sugar acids.

A “phosphate” moiety refers to a PO₄ group wherein the bond to the parent moiety is through the oxygen atoms.

In particular, exemplary functional groups include, but are not limited to:

Ethyleneimine, aniline-boronic acid, diethyl ester, 2,5-dimercaptoterephthalic acid, n-(3-tifluoroethanesulfonyloxypropyl)-anthraquinone-2-carboxamide, thiophene, 1-[4-(4-dimethylamino-phenylazo)-3-[3,5-bis[3,5-bis[3,5-bis(3-butene-1-oxy)benzyloxy]benzyloxy]benzyloxy]phenyl]-2,2,2 trifluoroethanone, permethylated α-cyclodextrin-6^{A}-monoalcohol nitrate, dinitrophenyl substituted β-cyclodextrin, β- and γ-CD bearing a 4-amino-7-nitrobenz-2-oxa-1,3-diazole, sulfated and carboxymethylated β-cyclodextrins, mono(6-cyclohexylamino-6-deoxy)-β-cyclodextrin, mono(6-benzyl-imino-6-deoxy)-β-cyclodextrin, mono [6-(o-aminophenyl)imino-6-deoxy]-β-cyclodextrin, mono [6-(p-aminophenyl)imino-6-deoxy]-β-cyclodextrin, mono [6-(α-naphthyl)imino-6-deoxy]-β-cyclodextrin, hexakis(6-O-benzoyl)-α-cyclodextrin, heptakis(2,3,6-tri-O-benzoyl)-β-cyclodextrin, hexakis(2,3-di-O-benzyl)-α-cyclodextrin, hexakis(6-β-benzoyl-2,3-di-O-benzyl)-α-cyclodextrin, 2- and 6-amino-β-cyclodextrin, and 2A,3A-alloepithio-2A,3A-dideoxy-β-cyclodextrin, and combinations thereof. In currently preferred embodiments, the functional groups which are used to modify the surface of the nanowires are selected from the group consisting of 4-(3-trifluoromethylazirino)benzoyl-N-succinimide (TDBA-OSu) and para-phenylenediamine (PPD).

Alternatively, the surface of the nanowires is modified with thin polymer films. According to currently preferred embodiments, the polymer films have thicknesses ranging from about 1 nm to about 500 nm. Various polymers are suitable within the scope of the present invention including, but not limited to, poly(3,4-ethylenedioxy)-thiophene-poly(styrene sulfonate) (PEDOT-PSS), poly(sulfone), poly(ethylene-co-vinyl acetate), poly(methyl methacrylate), tributyl phosphate (TBP), tricresyl phosphate, polyaniline, poly(vinylpyrrolidone), polycaprolactone, hydroxypropylcellulose, poly(ethyleneimine), tetracosanoic acid, tetraoctylammonium bromide, lauric acid, propyl gallate, quinacrine dihydrochloride dehydrate, quinacrine dihydrochloride, and the like.

Functionalizing the Si NW FETs can be performed by several procedures, of which non-limiting examples are described hereinbelow.

Functionalization through Chlorination Route

Chlorinated Si(111) surfaces can be prepared in two different methods. In one chlorination method, an H-terminated sample is immersed into a saturated solution including PCl₅, PBr₅, and PI₅ that contains a few grains of radical initiator, e.g. C₆H₅OOC₆H₅. The reaction solution is heated to 90-100° C. for 45 minutes. In another chlorination method, an H-terminated sample is placed into a Schlenk reaction tube and transported to a vacuum line. Approximately 50-200 Ton of Cl₂(g) is introduced through the vacuum line into the reaction tube, and the sample is illuminated for 30 seconds with 366 nm ultraviolet light. Excess Cl₂(g) is then removed under vacuum, and the flask is transported to the N₂(g)-purged flush box. Functionalizing the Si surfaces is performed by immersion in 1.0-3.0 molar R—MgX, wherein R represents the molecular backbone, and X═Cl, Br, or I. The duration of the reaction is approximately 1.5-16 hours at 70-80° C., wherein longer and bulkier molecular chains require longer reaction times. Excess THF, or other pertinent organic solvent, is added to all reaction solutions for solvent replacement. At the end of the reaction, the samples are removed from the reaction solution and then rinsed in THF, CH₃OH, and occasionally TCE. Samples are then sonicated for about 5 minutes in CH₃OH and CH₃CN and subsequently dried.

Functionalization by Lewis Acid-Mediated Terminal Alkenyl Reduction

Freshly etched, H-terminated Si (111) surfaces are functionalized by immersing approximately equal volumes of the molecule of interest with 1.0 M C₂H₅AlCl₂ in hexane at room temperature for 12 hours. Samples are removed from solution and rinsed in THF, CH₂Cl₂, and CH₃OH, and then dried.

Functionalization by Electrochemical Reduction of R—MgI

Samples are mounted to a cell to perform surface functionalization reactions. The samples are etched by filling the cell with 40% NH₄F(aq). After 20 minutes, the etching solution is removed and the cell is filled with H₂O to rinse the sample surface. The H₂O is then removed from the cell, and the sample is dried under a stream of N₂(g). The cell is then moved into the N₂(g)-purged flush box for electrochemical modification. Each chamber of the electrochemical cell contains a section of Cu gauze that serves as a counter electrode. A single counter electrode is produced. Molecular modification is performed in 3.0 M CH₃MgI in diethyl ether by applying 0.1 mA cm⁻² of constant anodic current density for 5 minutes with continuous stirring of the solution. After surface modification, the cell is rinsed with CH₂Cl₂ and CH₃OH. The cell is then dismantled, and the top and bottom ohmic contacts are scribed off to leave behind the portion of the wafer that had been exposed to the reaction solution solely. This wafer is re-rinsed in CH₃OH, sonicated in CH₃OH, further sonicated in CH₃CN, and dried with a stream of N₂(g).

Functinalization with Polymer Films

Polymer films are grown via layer-by-layer or ring-opening metathesis polymerization approaches according to procedures well known in the art. Attachment of the polymers mentioned herein to the Si NW surface can be done via ruthenium ring-opening metathesis polymerization catalyst as described in Juang et al. (Langmuir 2001, 17: 1321-1323). Briefly, The Si samples are etched with HF and optionally further etched with NH₄F. The resulting H-terminated Si surface is then chlorinated by exposure to saturated PCl₅ in chlorobenzene (45 minutes; 90-100° C.), with a trace of benzoyl peroxide added to serve as a radical initiator. The chloride capped Si surface is then exposed to allylmagnesium chloride for 14-16 hours at 75° C. in THF. An olefin metathesis catalyst (Cy₃P)₂Cl₂Ru═CHPh, wherein Cy=cyclohexyl is then reacted with the olefin-modified Si surface by immersing the Si for 3 hours into a 25 mM solution of (Cy₃P)₂Cl₂Ru═CHPh in CH₂Cl₂. The substrate is then rinsed several times with CH₂Cl₂ to remove any unbound catalyst. Exposure of the surface-bound catalyst to a solution of monomers of the desired polymer immersed in suitable solvent results in the growth of polymeric films on the Si surface. In this manner, control over the thickness of the polymer attached to the silicon substrate from sub-nanometers to hundreds of nanometers is achieved.

### Analysis

According to one embodiment, a method to determine the composition and concentration of volatile explosive compounds in a sample, comprising exposure of the sensors of the apparatus to the sample and using pattern recognition algorithms in order to identify and possibly quantify desired explosives in a given sample is provided in the present invention. Thus, the apparatus of the present invention further includes a pattern learning and recognition analyzer. In practice, the analyzer receives output signals from the device and analyses them by various pattern analysis algorithms to produce an output signature. By comparing an unknown signature with a database of stored or known signatures, explosive compounds can be identified.

Various analyses suitable for identifying and preferably quantifying volatile explosive compounds include, but are not limited to, principal component analysis, Fischer linear analysis, neural networks, genetic algorithms, fuzzy logic, pattern recognition, and other algorithms. After analysis is completed, the resulting information is displayed on display or transmitted to a host computer.

Many of the algorithms are neural network based algorithms. A neural network has an input layer, processing layers and an output layer. The information in a neural network is distributed throughout the processing layers. The processing layers are made up of nodes that simulate the neurons by the interconnection to their nodes.

In operation, when a neural network is combined with a sensor array, the sensor data is propagated through the networks. In this way, a series of vector matrix multiplications are performed and unknown analytes can be readily identified and determined. The neural network is trained by correcting the false or undesired outputs from a given input. Similar to statistical analysis revealing underlying patterns in a collection of data, neural networks locate consistent patterns in a collection of data, based on predetermined criteria.

Suitable pattern recognition algorithms include, but are not limited to, artificial neural networks including, but not limited to, multi-layer perception (MLP), generalized regression neural network (GRNN), fuzzy inference systems (FIS), self-organizing map (SOM), radial bias function (RBF), genetic algorithms (GAS), neuro-fuzzy systems (NFS), and adaptive resonance theory (ART). In other embodiments, the algorithms comprise statistical methods including, but not limited to, principal component analysis (PCA), partial least squares (PLS), multiple linear regression (MLR), principal component regression (PCR), discriminant function analysis (DFA) including linear discriminant analysis (LDA), and cluster analysis including nearest neighbor.

In currently preferred embodiments, principal component analysis is used. Principal component analysis (PCA) involves a mathematical technique that transforms a number of correlated variables into a smaller number of uncorrelated variables. The smaller number of uncorrelated variables is known as principal components. The first principal component or eigenvector accounts for as much of the variability in the data as possible, and each succeeding component accounts for as much of the remaining variability as possible. The main objective of PCA is to reduce the dimensionality of the data set and to identify new underlying variables.

In practice, PCA compares the structure of two or more covariance matrices in a hierarchical fashion. For instance, one matrix might be identical to another except that each element of the matrix is multiplied by a single constant. The matrices are thus proportional to one another. More particularly, the matrices share identical eigenvectors (or principal components), but their eigenvalues differ by a proportional constant. Another relationship between matrices is that they share principal components in common, but their eigenvalues differ. The mathematical technique used in PCA is called eigen analysis. The eigenvector associated with the largest eigenvalue has the same direction as the first principal component. The eigenvector associated with the second largest eigenvalue determines the direction of the second principal component. The sum of the eigenvalues equals the trace of the square matrix and the maximum number of eigenvectors equals the number of rows of this matrix.

### Applications

The present invention provides a method to detect volatile compounds derived from explosive materials in a sample, comprising exposing the sensors of the apparatus to a sample and using pattern recognition algorithms in order to identify and possibly quantify the components of the sample.

In one embodiment, the present invention is used to detect minute concentrations of explosive vapors. In a currently preferred embodiments, the detection of volatile compounds derived from explosive materials is performed with sensitivity below one part per million (ppm). More preferably, the apparatus and system of the present invention detect volatile compounds with sensitivity of less than 100 parts per billion (ppb). Most preferably, the apparatus and system of the present invention detect volatile compounds with sensitivity of one part per billion (ppb) or less.

According to one embodiment, the Si NW sensors possess the FET-like structures. These field effect transistors are normally used for sensing chemical processes, also known as CHEMFETs. There are many different varieties of CHEMFETS, most of which are based on a common principle, namely the presence of molecules or ions affect the potential of the conducting FET channel either by directly influencing the gate potential (e.g., for a catalytically active metal gate) or by changing the potential distribution between a “reference electrode gate” and the semiconductor. Since infinitesimal chemical perturbations can result in large electrical response, Si NW sensors are sensitive to, and can be used to detect, minute concentrations of chemicals. Without being bound by any theory or mechanism of action, the Si NW sensors used along with a reference gate and an ideal polar layer, induce a significant field in the channel. This field ensues due to the overall potential difference between the ground and reference electrodes. Thus, the field is induced to compensate for the potential drop.

According to other embodiments, chemical sensing can be produced using Si NW FETs with no reference electrode. Such devices have generally been referred to as molecularly controlled semiconductor resistors (MOCSERs). In MOCSERs, the traditional gating electrode is either present at the back, with a molecular layer adsorbed directly on the semiconductor, or is replaced altogether by a molecular layer adsorbed on a (typically ultra-thin) dielectric. Without being bound by any theory or mechanism of action, in either one of said configurations binding of molecules from the gas or liquid phase to the “chemical sensing molecules” changes the potential in the conducting channel. Consequently, the current between source and drain is modified and the device serves as a sensor. Such devices can have high chemical sensitivity.

According to the principles of the present invention FET-like structures further comprise ion selective field effect transistor (ISFET), surface accessible field effect transistor (SAFET), or suspended gate field effect transistor (SGFET).

In some embodiments, the apparatus and system of the present invention comprise sensors which are designed to detect vapors of explosive compounds. In other embodiments, the apparatus and system of the present invention comprise sensors which are designed to detect decomposition fragments of explosive compounds. In yet other embodiments, said apparatus and system comprise sensors which are designed to detect nitro-based explosives. In particular embodiments the apparatus and system of the present invention are designed to detect vapors derived from explosive materials including, but not limited to, nitric oxide (NO) and/or nitric dioxide (NO₂) gases.

In particular embodiments, the apparatus and system of the present invention is designed to detect minute concentration of explosive materials and vapors thereof selected from the group consisting of: pentaerythitol tetranitrate (PETN), tetranitro-tetrazacylooctane (HMX), nitroglycerin (NG), ethylene glycol dinitrate (EGDN), NH₄NO₃, dinitrotoluene (DNT), trinitrotoluene (TNT), tetryl, picric acid, and cyclotrimethylenetrinitramine (RDX). In a currently preferred embodiment, the apparatus and system of the present invention detects mixtures of explosives including, but not limited to, the mixtures disclosed herein in table 1.

Due to the miniaturized dimensions of the apparatus (in the range of 2-120 nanometers to a few micrometers), it could be installed in any electronic device. For example, these apparatuses could be integrated in a watch or cellular phone. The miniature size in which the apparatuses of the present invention can be produced, allows for their use as a warning system which provides detection unrevealed to the surroundings.

It should be noted that, as used in this specification and the appended claims, the singular forms “a,” “an,” and “the” include plural referents unless the content clearly dictates otherwise. Thus, for example, reference to “an amine moiety” may include two or more amine moieties. It should also be noted that the term “or” is generally employed in its sense including “and/or” unless the content clearly dictates otherwise.

The principles of the present invention are demonstrated by means of the following non-limitative examples.

### Examples

### Example 1

### Synthesis of the Silicon Nanowires (Si NWs)

The synthesis of Si NWs was performed as described in WO 2009/013754 which is incorporated herein by reference in its entirety. In particular, Si NWs were prepared by the vapor-liquid-solid (VLS) growth method using chemical vapor deposition (CVD) with silane on Si(111) substrates. Si substrates were etched in diluted HF to remove the native oxide following by sputtering of a 2 nm thick Au film on the substrate. The sample was transferred into the CVD chamber, and annealed at ˜580° C. with a pressure of ˜5×10⁻⁷ mbar for 10 minutes. The temperature was then dropped to ˜520° C. and a mixture of 5-10 sccm Ar and 5 sccm SiH₄ was introduced for 20 minutes at a pressure of 0.5-2 mbar to obtained undoped Si NWs.

Doped Si NWs were prepared by the vapor-liquid-solid (VLS) growth technique under gas ratios of 10 seem He, 5 seem SiH₄, and 0.02 seem B₂H₆ (2% in He), yielding p-type Si NWs doped with Boron. TEM characterization indicated that these NWs are essentially smooth having a diameter of 52±8 nm. The surface of the Si NW was covered with native oxide and minute amounts of gold.

### Example 2

### Functionalization of Si Nanowires (Si NWs)

The functionalization of the Si NWs was performed as described in WO 2009/013754 which is incorporated herein by reference in its entirety. In particular, functionalization of the Si NWs of the present invention was performed using a two-step chlorination/alkylation route. Prior to any chemical treatment, each sample was cleaned using a nitrogen (N_{2(g)}) flow. Hydrogen-terminated Si NWs were then prepared by etching the amorphous SiO₂ coating. This was done through exposing the Si NWs to buffered HF solution (pH=5) for 60 seconds followed by exposure to NH₄F for 30 seconds. It is noteworthy that longer exposures to HF and/or NH₄F results in fluorination of the sample thus interfering with the alkylation process. The sample was then removed and rinsed in water for <10 seconds per each side to limit oxidation, and dried in N_{2(g)} flow for 10 seconds. The sample was transferred into a glove-box with N_{2(g)}-atmosphere for functionalization.

Functionalization was preformed by immersing the sample into a saturated solution of PCl₅ in C₆H₅Cl (0.65M) that contains a few grains of C₆H₅OOC₆H₅ to act as a radical initiator (Hassler and Koell, J. Organometal. Chem. 1995, 487: 223). The reaction solution was heated to 90-100° C. for 5 minutes. The sample was then removed from the reaction solution and rinsed in tetrahydrofuran (THF) followed by a methanol (CH₃OH) rinse and drying under a stream of N_{2(g)}. Additionally, several samples were further rinsed with 1,1,1-trichloroethane (TCE) before drying under N_{2(g)} flow. The chlorine-terminated Si NWs were alkylated by immersion in 0.5M alkyl Grignard in THF (RMgCl: where R represents an alkyl chain with 1-7 carbon atoms). The reaction was performed for 30-250 minutes at 80° C. Excess THF was added to all reaction solutions for solvent replacement. At the end of the reaction, the sample was removed from the reaction solution and was then rinsed in THF, methanol, and occasionally TCE. The sample was then dried under a stream of N₂(_{g}).

### Example 3

### Fabrication of the Si NW Field Effect Transistors

The fabrication of the Si NW FETs was performed as described in WO 2009/013754 which is incorporated herein by reference in its entirety. In particular, devices were fabricated by depositing four Al electrodes on an individual Si NW on top of a 90 nm thermally oxidized degenerately doped p-type Si (0.001 Ω·cm⁻¹) substrate. The electrodes were mutually separated by 1.70±0.05 μm (FIG. 10). For each Si NW field effect transistor device, the intrinsic conductivity at determined back gate voltage was obtained by the four-point probe method. Particularly, electrical properties collected with the four-point probe method enable the configuration wherein there is no contact resistance between the metallic contacts and the Si NW.

### Example 4

### Functionalization of Si NWs with TDBA-OSu and PPD

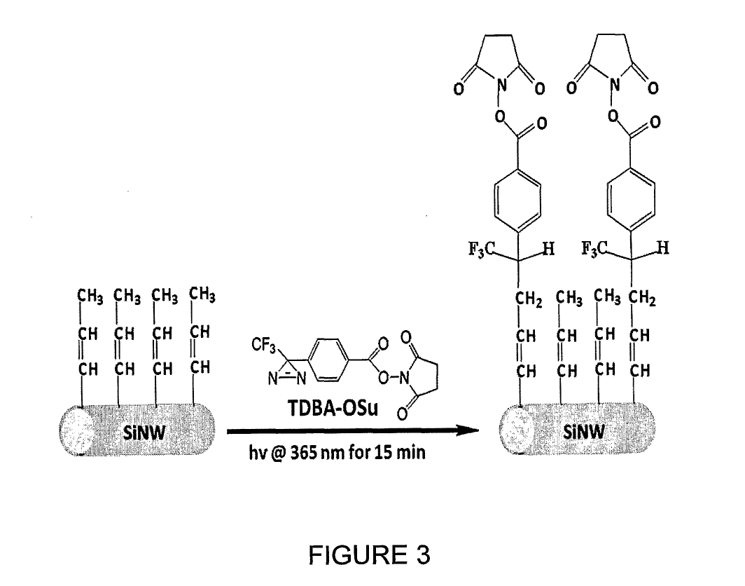

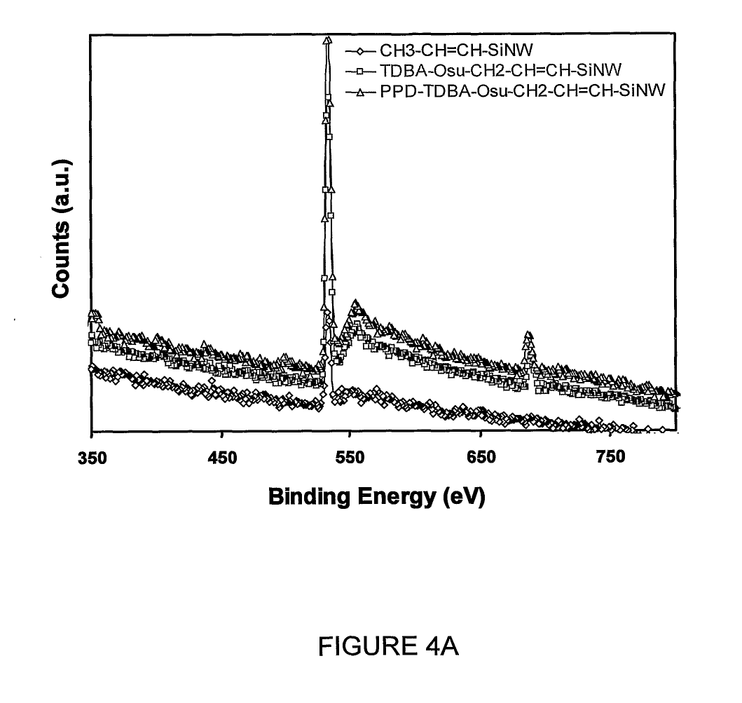

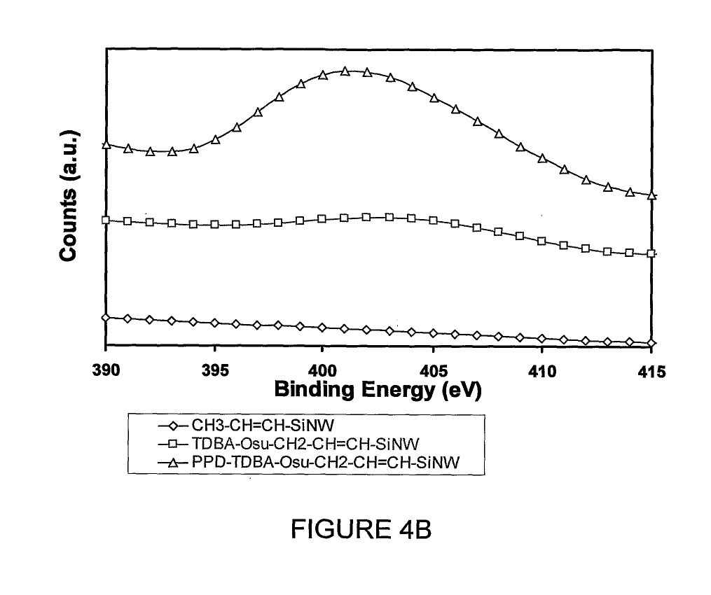

Si NWs (50 nm in diameter) samples with propenyl monolayers were placed in a 10-mm quartz cuvette. Then 0.2 mL of a 15 mM solution of 4-(3-trifluoromethylazirino)benzoyl-N-succinimide (TDBA-OSu) in dry CCl₄ was added and immediately illuminated with a broadband 365 nm UV lamp at a distance of 4 cm for 15 minutes. The samples were then rinsed vigorously with CCl₄, CH₂Cl₂, and water. After attachment of TDBA-OSu to Si—CH═CH—CH₃ (see FIG. 3), X-ray Photoelectron Spectroscopy (XPS) measurements showed the appearance of the F1s peak at around 680.0 eV due to the trifluoromethyl group of the TDBA-OSu cross-linker (FIG. 4A).

The C1s signal of the TDBA-OSu-CH₂—CH═CH—Si NW surfaces can be deconvoluted into four peaks, as follows: (i) a peak at 284.0 eV for carbon atoms that are covalently bonded to silicon (C—Si); (ii) a peak at 285.0 eV for carbons in the aliphatic hydrocarbon chain; (iii) a peak at 286.6 eV for α-carbons adjacent to the carbonyl carbon atoms; and (iv) a peak at 289.1 eV for the carbonyl carbon atoms. The ratio between these four peaks (1.4:6.5:2.6:1), before and after 10 minutes sonication, was found to be equivalent to ˜50% density of reactive amino groups on the surface. XPS spectra of the Si2p region showed no surface oxidation before and after the secondary functionalization process, indicating that the propenyl monolayer was not damaged during the secondary functionalization and has no degradation effects on the stability of the TDBA-OSu-CH₂—CH═CH—Si NWs. TDBA-OSu-CH₂—CH═CH—Si NW samples were subsequently placed in a solution of 5 mM para-phenylenediamine (PPD) in DMF. After immersing for 2 hours, the samples were cleaned by extensive rinsing with DMF and CH₂Cl₂, and dried using N₂(g) flushing. This process provided para-phenylenediamine (PPD) on TDBA-OSu-CH₂—CH═CH—Si NWs.

The XPS spectra of PPD-TDBA-OSu-CH₂—CH═CH—Si samples, before and after 10 minutes sonication, indicated that the CH₃—CH═CH—Si layer was intact and that the TDBA-OSu and PPD functionalities were covalently bonded to Si NW surfaces (FIG. 4A). The main peak of the XPS spectra of TDBA-OSu-CH₂—CH═CH—Si and PPD-TDBA-OSu-CH₂—CH═CH—Si is assigned to O1s. XPS spectra at 400 eV showed the existence of the N1s peaks due to the low atomic ratio of nitrogen in TDBA-OSu and relatively large atomic ratio in PPD (FIG. 4B). Thus it is clearly shown that the TDBA-OSu and PPD are covalently attached to the CH₃—CH═CH—Si NW. F1s peak showed no difference before and after sonication of TDBA-OSu-CH₂—CH═CH—Si or PPD-TDBA-OSu-CH₂—CH═CH—Si samples indicating that the CH₃—CH═CH—Si layer is intact and that the TDBA-OSu and PPD are covalently bonded. Further reacting CH₃—Si NWs with TDBA-OSu according to the abovementioned scheme showed no evidence for the presence of subsequent covalent functionalization.

### Example 5

### Sensor Measurements

Sensor measurements using the Si NW FETs of the present invention were performed as described in WO 2009/013754 which is incorporated herein by reference in its entirety. The developed sensors were placed in a 316-stailnless steel chamber with PTFE O-rings. To assess the sensing characteristics of the various Si NWs, current-voltage measurements at determined back gate voltage of each sensor were performed with digital multimeter (model 34411A; Agilent Technologies Ltd.) that is multiplexed with 40-channel armature multiplexer (model 34921A; Agilent Technologies Ltd.). In these measurements, a voltage of −3 V was applied to the degeneratively doped silicon substrate that was coated with 200 nm aluminum, as an ohmic contact. The −3 V back-gate-voltage value was chosen to provide an optimal signal-to-noise ratio of the output signal. Under this value of back gate voltage, four-point probe transport measurements were carried out, at bias range between −5 and +5 V, in steps of 10 mV, with the two inner electrodes serving as voltage probes and the two outer electrodes serving as current probes.

A Labview-controlled automated flow system delivered pulses of desired vapors at a controlled vapor pressure optimized to the detector surface area. Dry air was obtained from a house compressed air source, controlled with a 10 L/minute mass flow controller. In a typical experiment, signals of sensor array elements were collected for 70 seconds of clean laboratory air, followed by 80 seconds of desired vapors in air, followed by another 70 seconds interval of clean air to purge the system. Data analysis of the signals collected from all the sensors in the array was performed using standard principal component analysis.

### Example 6

### Detection of NO and NO₂ Using the Sensors of the Present Invention

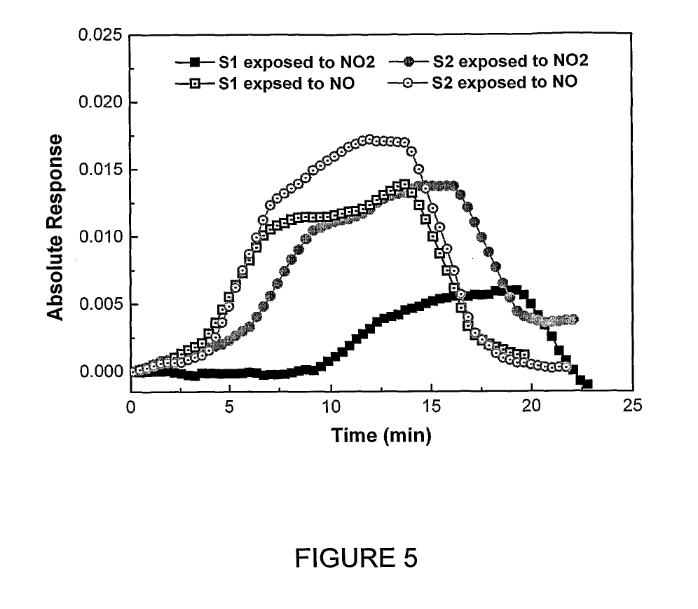

TDBA-OSu-CH₂—CH═CH—Si NW FETs (hereinafter, Si) and PPD-TDBA-OSu-CH₂—CH═CH—Si NW FETs (hereinafter, S2) were exposed at each time to 10 ppb of NO and NO₂ (FIG. 5). NO and NO₂ were chosen to simulate explosive detection since these gases are the main products emitted from explosive materials. Therefore, detecting NO and NO₂ is indicative for the presence of explosives.

The Exposure of S1 to 10 ppb NO₂ provided a robust signal-to-noise ratio, with slow sensing response (FIG. 5; full squares). Exposure of the same sensor to 10 ppb NO provided higher sensing response and, additionally, shorter response time (FIG. 5; empty squares).

Adding amine functionalities to the surface of Si NWs, via the PPD molecules (see S2), enhanced the sensitivity to both NO and NO₂ molecules. As could be seen in FIG. 5, S2 provided high sensing response to both NO₂ (full circles) and NO (empty circles) with slightly enhanced response for the latter. Importantly, the sensing response of S2 was significantly higher, as compared to S1, when exposed to either NO₂ or NO. This could be attributed to the addition of the PPD (or amine) functionalities which enhance the adsorption of the NO or NO₂ molecules to the Si NWs surface. Without being bound by any theory or mechanism of action, the better responses of both sensors to NO in comparison to NO₂ might be attributed to better absorption of NO molecules within the functionalized layer and/or to the higher dipole moment of NO in comparison to NO₂ molecules. It is contemplated that the adsorbed NO molecules on the NW surface induce higher electrostatic field into the Si NW and therefore result in higher sensing signal.

These results clearly demonstrate the compatibility of these sensors to detect explosive materials and volatile compounds derived from explosive materials. The sensing of NO and NO₂ which are major constituents of vapors derived from explosives is obtained with very high sensitivity, wherein a concentration of 10 ppb induced a detectable sensor response. Furthermore, the results demonstrate the importance of functionalizing the Si NW FETs with particular organic functionalities (amine moieties) for enhancing the sensitivity of the Si NW FETs to nitro-compounds derived from explosive materials.

While the present invention has been particularly described, persons skilled in the art will appreciate that many variations and modifications can be made. Therefore, the invention is not to be construed .as restricted to the particularly described embodiments, and the scope and concept of the invention will be more readily understood by reference to the claims, which follow.

1. An apparatus for detecting volatile compounds derived from explosive materials, comprising at least one chemically sensitive sensor comprising field effect transistors of non-oxidized, silicon nanowires functionalized with at least one of an amine, an imine, an amide, an ammonium, a keto, an alcohol, a phosphate, a thiol, a sulfonate, a sulfonyl or a carboxyl moiety.

2. (canceled)

3. (canceled)

4. The apparatus according to claim 1, wherein the silicon nanowires are functionalized with at least one moiety selected from the group consisting of carboxyalkyl, carboxycycloalkyl, carboxyalkenyl, carboxyalkynyl, carboxyaryl, carboxyheterocyclyl, carboxyheteroaryl, carboxyalkylaryl, carboxyalkylalkenyl, carboxyalkylalkynyl, carboxyalkylcycloalkyl, carboxyalkylheterocyclyl carboxyalkyiheteroaryl, alkylamine, cycloalkylamine, alkenylamine, alkynylamine, arylamine, heterocyclylamine, heteroarylamine, alkylarylamine, alkylalkenylamine, alkylalkynylamine, alkylcycloalkylamine, alkylheterocyclylamine alkylheteroarylamine, alkylimine, cycloalkylimine, alkenylimine, alkynylimine, arylimine, heterocyclylimine, heteroarylimine, alkylarylimine, alkylalkenylimine, alkylalkynylimine, alkylcycloalkylimine, alkylheterocyclylimine, alkylheteroarylimine and combinations and derivatives thereof.

5. The apparatus according to claim 1, wherein the silicon nanowires are functionalized with at least one of ethyleneimine, aniline-boronic acid, diethyl ester, 2,5-dimereaptoterephthalic acid, n-(3-trifluoroethanesulfonyloxypropyl)-anthraquinone-2-carboxamide, thiophene, 1-[4-(4-dimethylamino-phenylazo)-3-[3,5-bis[3,5-bis[3,5-bis(3-butene-1-oxy)benzyloxy]benzyloxy]benzyloxy]phenyl]-2,2,2 trifluoroethanone, permethylated α-cyclodextrin-6^{A}-monoalcohol nitrate, dinitrophenyl substituted β-cyclodextrin, β- and γ-CD bearing a 4-amino-7-nitrobenz-2-oxa-1,3-diazole, sulfated and carboxymethylated β-cyclodextrins, mono(6-cyclohexylamino-6-deoxy)-β-cyclodextrin, mono(6-benzyl-imino-6-deoxy)-β-cyclodextrin, mono[6-(o-aminophenyl)imino-6-deoxy]-β-cyclodextrin, mono[6-(p-aminophenyl)imino-6-deoxy]-β-cyclodextrin, mono[6-(α-naphthyl)imino-6-deoxy]-β-cyclodextrin, hexakis(6-O-benzoyl)-α-cyclodextrin, heptakis(2,3,6-tri-O-benzoyl)-β-cyclodextrin, hexakis(2,3-di-O-benzyl)-α-cyclodextrin, hexakis(6-O-benzoyl-2,3-di-O-benzyl)-α-cyclodextrin, 2- and 6-amino-β-cyclodextrin, 2A,3A-alloepithio-2A,3A-dideoxy-β-cyclodextrin, and combinations thereof.

6. The apparatus according to claim 1, wherein the silicon nanowires are functionalized with 4-(3-trifluoromethylazirino)benzoyl-N-succinimide (TDBA-OSu), para-phenylenediamine (PPD) or a combination thereof.

7. The apparatus according to claim 1, wherein the silicon nanowires are functionalized with a thin polymer film selected from poly(3,4-ethylenedioxy)-thiophene-poly(styrene sulfonate) (PEDOT-PSS), poly(sulfone), poly(ethylene-co-vinyl acetate), poly(methyl methacrylate), tributyl phosphate (TBP), tricresyl phosphate, polyaniline, poly(vinylpyrrolidone), polycaprolactone, hydroxypropylcellulose, poly(ethyleneimine), tetracosanoic acid, tetraoctylammonium bromide, lauric acid, propyl gallate, quinacrine dihydrochloride dehydrate, and quinacrine dihydrochloride.

8. The apparatus according to claim 1, wherein the explosive materials to be detected are selected from the group consisting of pentaerythitol tetranitrate (PETN), tetranitro-tetrazacylooctane (HMX), nitroglycerin (NG), ethylene glycol dinitrate (EGDN), NH₄NO₃, dinitrotoluene (DNT), trinitrotoluene (TNT), tetryl, picric acid, cyclotrimethylenetrinitramine (RDX), mixtures and fragments thereof.

9. A system having:
(i) an apparatus for detecting volatile compounds derived from explosive materials, wherein the apparatus comprises an array of chemically sensitive sensors comprising field effect transistors (FETs) of non-oxidized, silicon nanowires (Si NW) functionalized with at least one of an amine, an imine, an amide, an ammonium, a keto, an alcohol, a phosphate, a thiol, a sulfonate, a sulfonyl or a carboxyl moiety, and
(ii) a learning and pattern recognition analyzer, wherein the learning and pattern recognition analyzer receives sensor output signals and compares them to stored data.

10. (canceled)

11. (canceled)

12. The system according to claim 9, wherein the silicon nanowires are functionalized with at least one moiety selected from the group consisting of carboxyalkyl, carboxycycloalkyl, carboxyalkenyl, carboxyalkynyl, carboxyaryl, carboxyheterocyclyl, carboxyheteroaryl, carboxyalkylaryl, carboxyalkylalkenyl, carboxyalkylalkynyl, carboxyalkylcycloalkyl, carboxyalkylheterocyclyl carboxyalkylheteroaryl, alkylamine, cycloalkylamine, alkenylamine, alkynylamine, arylamine, heterocyclylamine, heteroarylamine, alkylarylamine, alkylalkenylamine, alkylalkynylamine, alkylcycloalkylamine, alkylheterocyclylamine alkylheteroarylamine, alkyl imine, cycloalkylimine, alkenylimine, alkynylimine, arylimine, heterocyclylimine, heteroarylimine, alkylarylimine, alkylalkenylimine, alkylalkynylimine, alkylcycloalkylimine, alkylheterocyclylimine, alkylheteroarylimine and combinations and derivatives thereof.

13. The system according to claim 9, wherein the silicon nanowires are functionalized with at least one of ethyleneimine, aniline-boronic acid, diethyl ester, 2,5-dimercaptoterephthalic acid, n-(3-trifluoroethanesulfonyloxypropyl)-anthraquinone-2-carboxamide, thiophene, 1-[4-(4-dimethylamino-phenylazo)-3-[3,5-bis[3,5-bis[3,5-bis(3-butene-1-oxy)benzyloxy]benzyloxy]benzyloxy]phenyl]-2,2,2 trifluoroethanone, permethylated α-cyclodextrin-6^{A}-monoalcohol nitrate, dinitrophenyl substituted β-cyclodextrin, β- and γ-CD bearing a 4-amino-7-nitrobenz-2-oxa-1,3-diazole, sulfated and carboxymethylated β-cyclodextrins, mono(6-cyclohexylamino-6-deoxy)-β-cyclodextrin, mono(6-benzyl-imino-6-deoxy)-β-cyclodextrin, mono[6-(o-aminophenyl)imino-6-deoxy]-β-cyclodextrin, mono[6-(p-aminophenyl)imino-6-deoxy]-β-cyclodextrin, mono[6-(α-naphthyl)imino-6-deoxy]-β-cyclodextrin, hexakis(6-O-benzoyl)-α-cyclodextrin, heptakis(2,3,6-tri-O-benzoyl)-β-cyclodextrin, hexakis(2,3-di-O-benzyl)-α-cyclodextrin, hexakis(6-O-benzoyl-2,3-di-O-benzyl)-α-cyclodextrin, 2- and 6-amino-β-cyclodextrin, 2A,3A-alloepithio-2A,3A-dideoxy-β-cyclodextrin, and combinations thereof.

14. The system according to claim 9, wherein the silicon nanowires are functionalized with 4-(3-trifluoromethylazirino)benzoyl-N-succinimide (TDBA-OSu), para-phenylenediamine (PPD) or a combination thereof.

15. The system according to claim 9, wherein the silicon nanowires are functionalized with a thin polymer film selected from, poly(3,4-ethylenedioxy)-thiophene-poly(styrene sulfonate) (PEDOT-PSS), poly(sulfone), poly(ethylene-co-vinyl acetate), poly(methyl methacrylate), tributyl phosphate (TBP), tricresyl phosphate, polyaniline, poly(vinylpyrrolidone), polycaprolactone, hydroxypropylcellulose, poly(ethyleneimine), tetracosanoic acid, tetraoctylammonium bromide, lauric acid, propyl gallate, quinacrine dihydrochloride dehydrate, and quinacrine dihydrochloride.

16. The system according to claim 9, wherein the explosive materials to be detected are selected from the group consisting of pentaerythitol tetranitrate (PSTN), tetranitro-tetrazacylooctane (HMX), nitroglycerin (NG), ethylene glycol dinitrate (EGDN), NH₄NO₃, dinitrotoluene (DNT), trinitrotoluene (TNT), tetryl, picric acid, cyclotrimethylenetrinitramine (RDX), mixtures and fragments thereof.

17. The system according to claim 9, wherein the learning and pattern recognition analyzer comprises at least one algorithm selected from the group consisting of artificial neural network algorithms, principal component analysis (PCA), multi-layer perception (MLP), generalized regression neural network (GRNN), fuzzy inference systems (FIS), self-organizing map (SOM), radial bias function (RBF), genetic algorithms (GAS), neuro-fuzzy systems (NFS), adaptive resonance theory (ART), partial least squares (PLS), multiple linear regression (MLR), principal component regression (PCR), discriminant function analysis (DFA), linear discriminant analysis (LDA), cluster analysis, and nearest neighbor.

18. (canceled)

19. A method for detecting volatile compounds derived from explosive materials in a sample, comprising the steps of:
i) providing a system comprising an apparatus for detecting volatile explosive compounds comprising an array of chemically sensitive sensors comprising field effect transistors of non-oxidized silicon nanowires functionalized with at least one of an amine, an imine, an amide, an ammonium, a keto, an alcohol, a phosphate, a thiol, a sulfonate, a sulfonyl or a carboxyl moiety, further comprising a learning and pattern recognition analyzer, wherein said learning and pattern recognition analyzer receives sensor output signals from the apparatus and compares them to stored data,
ii) exposing the sensor array of said apparatus to the sample, and
iii) using pattern recognition algorithms to detect the presence of volatile compounds derived from explosive materials in the sample.

20. (canceled)

21. (canceled)

22. The method according to claim 19, wherein the silicon nanowires are functionalized with at least one moiety selected from the group consisting of carboxyalkyl, carboxycycloalkyl, carboxyalkenyl, carboxyalkynyl, carboxyaryl, carboxyheterocyclyl, carboxyheteroaryl, carboxyalkylaryl, carboxyalkylalkenyl, carboxyalkylalkynyl, carboxyalkylcycloalkyl, carboxyalkylheterocyclyl carboxyalkylheteroaryl, alkylamine, cycloalkylamine, alkenylamine, alkynylamine, arylamine, heterocyclylamine, heteroarylamine, alkylarylamine, alkylalkenylamine, alkylalkynylamine, alkylcycloalkylamine, alkylheterocyclylamine alkylheteroarylamine, alkylimine, cycloalkylimine, alkenylimine, alkynylimine, arylimine, heterocyclylimine, heteroarylimine, alkylarylimine, alkylalkenylimine, alkylalkynylimine, alkylcycloalkylimine, alkylheterocyclylimine, alkylheteroarylimine and combinations and derivatives thereof.

23. The method according to claim 19, wherein the silicon nanowires are functionalized with at least one of ethyleneimine, aniline-boronic acid, diethyl ester, 2,5-dimercaptoterephthalic acid, n-(3-trifluoroethanesulfonyloxypropyl)-anthraquinone-2-carboxamide, thiophene, 1-[4-(4-dimethylamino-phenylazo)-3-[3,5-bis[3,5-bis[3,5-bis(3-butene-1-oxy)benzyloxy]benzyloxy]benzyloxy]phenyl]-2,2,2 trifluoroethanone, permethylated α-cyclodextrin-6^{A}-monoalcohol nitrate, dinitrophenyl substituted β-cyclodextrin, and γ-CD bearing a 4-amino-7-nitrobenz-2-oxa-1,3-diazole, sulfated and carboxymethylated β-cyclodextrins, mono(6-cyclohexylamino-6-deoxy)-β-cyclodextrin, mono(6-benzyl-imino-6-deoxy)-β-cyciodextrin, mono[6-(o-aminophenyl)imino-6-deoxy]-β-cyclodextrin, mono[6-(p-aminophenyl)imino-6-deoxy]-β-cyclodextrin, mono[6-(α-naphthyl)imino-6-deoxy]-β-cyclodextrin, hexakis(6-O-benzoyl)-α-cyclodextrin, heptakis(2,3,6-tri-O-benzoyl)-β-cyclodextrin, hexakis(2,3-di-O-benzyl)-α-cyclodextrin, hexakis(6-O-benzoyl-2,3-di-O-benzyl)-α-cyclodextrin, 2- and 6-amino-β-cyclodextrin, 2A,3A-alloepithio-2A,3A-dideoxy-β-cyclodextrin, and combinations thereof.

24. The method according to claim 19, wherein the silicon nanowires are functionalized with 4-(3-trifluoromethylazirino)benzoyl-N-succinimide (TDBA-OSu), para-phenylenediamine (PPD) or a combination thereof.

25. The method according to claim 19, wherein the silicon nanowires are functionalized with a thin polymer film selected from poly(3,4-ethylenedioxy)-thiophene-poly(styrene sulfonate) (PEDOT-PSS), poly(sulfone), poly(ethylene-co-vinyl acetate), poly(methyl methacrylate), tributyl phosphate (TBP), tricresyl phosphate, polyaniline, poly(vinylpyrrolidone), polycaprolactone, hydroxypropylcellulose, poly(ethyleneimine), tetracosanoic acid, tetraoctylammonium bromide, lauric acid, propyl gallate, quinacrine dihydrochloride dehydrate, and quinacrine dihydrochloride.

26. The method according to claim 19, wherein the learning and pattern recognition analyzer comprises at least one algorithm selected from the group consisting of artificial neural network algorithms, principal component analysis (PCA), multi-layer perception (MLP), generalized regression neural network (GRNN), fuzzy inference systems (FIS), self-organizing map (SOM), radial bias function (RBF), genetic algorithms (GAS), neuro-fuzzy systems (NFS), adaptive resonance theory (ART), partial least squares (PLS), multiple linear regression (MLR), principal component regression (PCR), discriminant function analysis (DFA), linear discriminant analysis (LDA), cluster analysis, and nearest neighbor.

27. (canceled)

28. The method according of claim 19, wherein the explosive materials to be detected are selected from the group consisting of pentaerythitol tetranitrate (PETN), tetranitro-tetrazacylooctane (HMX), nitroglycerin (NG), ethylene glycol dinitrate (EGDN), NH₄NO₃, dinitrotoluene (DNT), trinitrotoluene (TNT), tetryl, picric acid, cyclotrimethylenetrinitramine (RDX), mixtures and fragments thereof.
